(19) United States
(12) Patent Application Publication
(10) Pub. No.: US 2008/0255550 A1
(43) Pub. Date: Oct. 16, 2008

(54) Systems and methods for less invasive neutralization by ablation of tissue including the appendix and gall bladder

(75) Inventor: Stephen Graham Bell, Roma (IT)

Correspondence Address: ROGITZ & ASSOCIATES, 750 B STREET, SUITE 3120, SAN DIEGO, CA, 92101, US

(73) Assignee: Minos Medical

(21) Appl. No.: 11/606,742

(22) Filed: Nov. 30, 2006

(51) Int. Cl. A61B 18/14; A61B 18/04; A61B 17/12; A61M 25/00; A61F 7/12

(52) U.S. Cl. 606/21; 606/34; 606/214; 604/21; 604/113

(57) ABSTRACT

Systems and methods for ablation of the gall bladder and appendix through a natural orifice.

### I. FIELD OF THE INVENTION

The present invention relates to less invasive surgical procedures, and more particularly to procedures that require no incision into the human body.

### II. BACKGROUND OF THE INVENTION

Less invasive procedures have been developed to resolve, e.g., by removal, maladies of tissue. An example of such a procedure is laparoscopy, in which a small incision is made near the navel and a device known as a laparoscope is inserted through the incision to view and/or remove tissue in the abdomen.

As understood herein, current less invasive procedures, while avoiding large incisions, nonetheless require incisions be made into the body through the abdominal wall, and any incision carries some degree of risk and patient discomfort. As further recognized herein, some commonly encountered maladies, including appendicitis and gall bladder derangements, can be surgically addressed without making any incision at all, but rather by advancing surgical instruments through a natural body orifice such as the anus or mouth (leading to the esophagus). The present invention still further recognizes, however, that aspects of such a procedure raise additional considerations that must also be addressed.

### SUMMARY OF THE INVENTION

A method is disclosed for neutralizing an organ such as the gall bladder or appendix without making an incision in the patient. The method includes advancing a catheter assembly into the intestines of a patient through a natural orifice (anus or esophagus) of the patient. A neutralization element of the catheter assembly is advanced into an organ of the patient and actuated to neutralize the organ from the interior thereof.

In one implementation, the neutralization element is an ablation catheter. An ablating fluid is infused into the organ through the ablation catheter. The ablating fluid may be, e.g., hot saline to thermally ablate the organ, or it may chemically ablate the organ. Or, the ablating fluid can be liquid such as saline that can be electrified to ablate the organ. Yet again, the ablating fluid can be a gas that can be electrified to ablate the organ. The ablating fluid may be Silver Nitrate.

If desired, the ablation catheter can include a balloon. When the organ is the gall bladder, a portion of the catheter can be advanced into the gall bladder with the balloon in the bile duct and inflated to seal the ablating fluid in the gall bladder.

In other implementations the neutralization element can be an ablation catheter having an expandable ablation member configured for ablating the organ. The ablation member may include a balloon inflatable with a fluid to substantially fill the organ, with the fluid being electrified to ablate the organ. Or, the ablation member may be an expandable metal mesh or array that can be electrified to ablate the organ.

In still other implementations the neutralization element can be an antenna. High intensity focused ultrasound (HIFU) energy can be transmitted to the antenna to ablate the organ.

In yet other implementations the neutralization element can be an adhesive infusion tube and adhesive can be infused into the organ through the tube to neutralize the organ.

In another aspect, a catheter assembly is advanced into the intestines of a patient through a natural orifice of the patient, and then a sealant element of the catheter is advanced into an organ of the patient. Sealant is infused through the sealant element into the organ to seal the organ.

In yet another aspect, a system for ablating the gall bladder or appendix include a catheter assembly that is advanceable into the intestines of a patient through a natural orifice of the patient. The system also includes an ablation catheter that is advanceable out of the catheter assembly into the gall bladder or appendix. Means are engaged with the ablation catheter for ablating the gall bladder or appendix or any other organ that can be reached via the natural orifice.

In still another aspect, a system for neutralizing the gall bladder or appendix includes a catheter assembly that is advanceable into the intestines of a patient through a natural orifice of the patient. A sealant catheter is advanceable out of the catheter assembly into the gall bladder or appendix, and a source of sealant is engaged with the catheter for infusing sealant through the catheter to seal the gall bladder or appendix.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a schematic diagram of a non-limiting catheter assembly advanced through a natural orifice into the gall bladder of a human patient;

FIG. 2 is a perspective view of a non-limiting catheter assembly showing an ablation catheter advanced out of an endoscope over a guidewire;

FIG. 3 is a cross-sectional diagram as seen along the line 3-3 in FIG. 2, with portions cut away for clarity;

FIG. 4 is a side view of an alternate ablation catheter disposed in the gall bladder;

FIG. 5 is a side view of the distal portion of a tissue grasper that can be advanced through the endoscope or the ablation catheter to grasp ablated tissue to invert it;

FIG. 6 is a side view of non-limiting inversion and excision elements that can be used to invert and ligate the gall bladder or appendix after ablation;

FIG. 7 is a side view of the inversion and excision elements with the organ inverted;

FIG. 8 is a side view of the inversion and excision elements with the organ inverted and the ligating loop closed;

FIGS. 9-13 are side views of the distal portions of an alternate ablation device that uses bipolar current through an inflatable balloon or metal sheath to ablate an organ such as an appendix;

FIGS. 14 and 15 are side views of the distal portions of an alternate ablation device that uses high intensity focused ultrasound (HIFU) to ablate an organ such as an appendix; and

FIGS. 16-19 are side views of the distal portions of an alternate device that infuses adhesive and used a vacuum to collapse and seal an organ such as the appendix to neutralize the organ.

### DETAILED DESCRIPTION OF THE PREFERRED EMBODIMENT

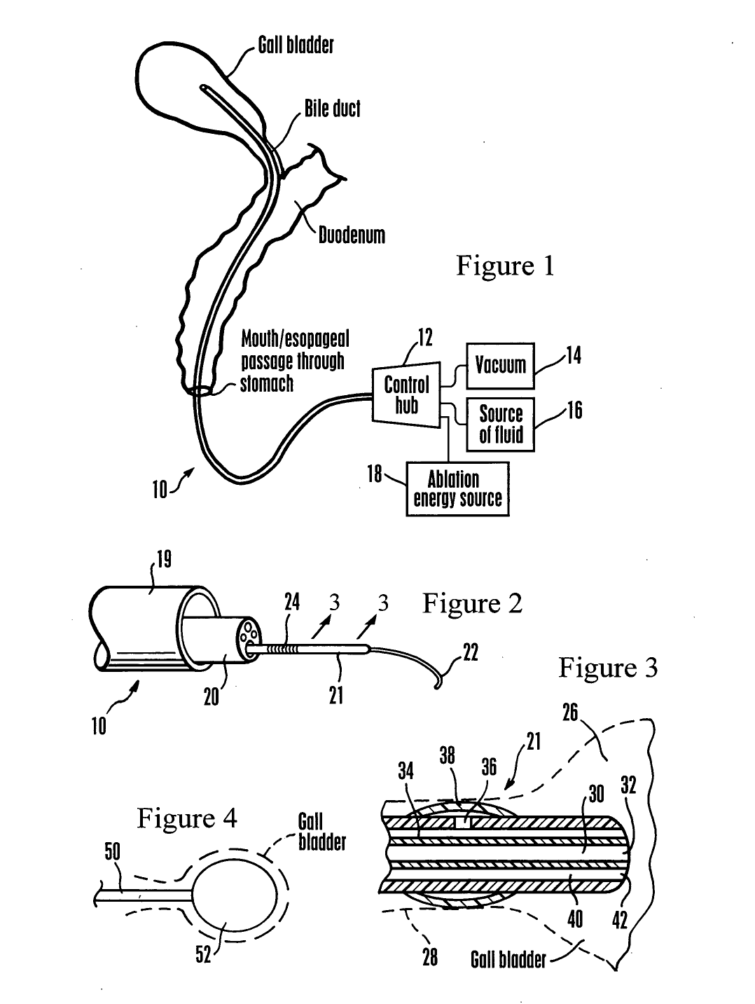

Referring initially to FIG. 1, a catheter assembly is shown, generally designated 10, for ablating and/or sealing an organ such as the gall bladder of a patient by advancing the assembly 10 through the mouth, esophagus, and duodenum and into, e.g., the bile duct and then the gall bladder and operating the assembly 10 as more fully disclosed below. In the case of the appendix, the catheter is advanced through the anus to the organ. Thus, the catheter assembly 10 is advanced into the patient through a natural orifice, i.e., through the anus or the esophagus, so that no incision need be made.

In the non-limiting implementation shown in FIG. 1, the catheter assembly 10 terminates in a control hub 12 outside the patient. Through the control hub 12, one or more lumens of the assembly 10 may be evacuated by a source 14 of vacuum. Also, a source 16 of ablating or sealing fluid can be connected to the hub 12 for infusing fluid through the catheter assembly 10 as more fully disclosed below. Moreover, a source 18 of ablation energy such as HIFU, bipolar electrical current, etc. may be connected to the hub 12 for activating the catheter assembly 10 to ablate tissue as more fully disclosed below.

With the overall environment of the catheter assembly 10 in mind, attention is now directed to FIG. 2. The non-limiting assembly 10 shown in FIG. 2 may include a hollow overtube 19 through which an elongated flexible catheter-like endoscope 20 can slide. In other embodiments no endoscope need be provided, with catheter guidance being effected by ultrasound or fluoroscopy as set forth further below. Or, the assembly 10 may not include an overtube 19 but only the endoscope 20 with catheters disclosed below being advanced out of the working channel of the endoscope. Even when an endoscope is provided, visualization can be provided using ultrasound or fluoroscopy.

As shown in FIG. 1, an ablation catheter 21 can be slidably disposed in a working channel of the endoscope 20. It is to be understood that other of the below-described catheters, including the sealant catheter and below-described HIFU catheter may likewise be slidably disposed in the working channel of the endoscope 20.

A guidewire 22 can be disposed in a lumen of the ablation catheter 21 and the catheter 21 slid over the guidewire 22. The guidewire 22 can be guided into the appendix or gall bladder by a surgeon viewing the organ using the endoscope 20 in conjunction with fluoroscopy or ultrasound or any other imaging modality such as magnetic resonance imaging or CT scan. Once the guidewire 22 is positioned in the organ, the ablation catheter 21 can be advanced into the organ and if desired radiopaque fluid infused into the appendix through the ablation catheter 21. In addition or in lieu of such infusion, the ablation catheter 21 can include axially-spaced radiopaque bands 24 that can be regarded as depth markings, for viewing of the ablation catheter 21 using fluoroscopy principles known in the art. Alternatively, ultrasonic imaging may be used. The guidewire 22 likewise can include axially-spaced radiopaque bands 24 that can be regarded as depth markings.

FIG. 3 shows how the ablation catheter 21 can be used to ablate the interior of an organ such as a gall bladder 26 that communicates with a bile duct 28. The catheter 21 includes one or more lumens, including an infusion lumen 30 that terminates in one or more infusion ports 32 that can be located at the distal tip of the catheter 21. It is to be understood that the infusion lumen 30 communicates with the source 16 of fluid shown in FIG. 1. Also, the catheter 21 may include an inflation port 34 communicating, via an inflation port 36, with the interior of an inflatable anchoring balloon 38 for inflating the anchoring balloon 38 with fluid while the balloon 38 is in the bile duct 28 as shown, sealing the gall bladder 26. It is to be understood that the anchoring balloon 38 is deflated to advance and remove the catheter 21. In some implementations a vacuum channel 40, which may communicate with the source 14 of vacuum in FIG. 1, can be provided to evacuate the organ through one or more vacuum ports 42.

It is to be understood that prior to ablation, the bile duct and gall bladder may be first cleared of stones using, e.g., the vacuum lumen 40 of the catheter 21 or using another instrument such as a stone removal cannula that is first advanced through the endoscope 20. Chemical destruction of gallstones followed by removal can also be effected.

It is to be further understood that prior to engaging the ablation catheter with the endoscope 20 and after positioning the endoscope through a natural orifice to locate its distal end adjacent the organ to be ablated, a fluidic substance can be infused through a channel of the endoscope 20 to destroy the mucus membrane of the organ. The fluid can then be evacuated through the endoscope. Alternatively, the ablation catheter can be advanced to the organ as described and the mucus membrane can be destroyed by infusing the fluidic substance through a lumen of the ablation catheter. Fluids for destroying mucus membranes are known in the art.

As yet another alternative, a coil or other electrocautery surface can be provided on the outside of the ablation catheter and energized once the catheter is positioned in the organ using bipolar or monopolar electrocautery principles known in the art to destroy by cautery the mucus membrane. As recognized herein, destruction of mucus membrane prior to ablation of the organ facilitates ablation.

In any case, in some instances without any preliminary steps or in other cases contemporaneous with or after gallstone removal and/or mucus membrane destruction as disclosed above, the catheter assembly 10 can be advanced, without making any incision, into the intestines of a patient through a natural orifice of the patient under, e.g., endoscopic guidance. The ablation catheter 21 then can be advanced into the gall bladder 26.

Once the gall bladder is cleared of stones (and in some cases with the mucus membrane of the gall bladder destroyed), the anchor balloon 38 can be inflated in the bile duct 28 to hold the catheter in place to isolate the gall bladder. Then, an ablating fluid can be infused into the organ through the infusion port or ports 32 of the ablation catheter. The ablating fluid can fill the organ to slightly distend it.

In one non-limiting implementation, the ablating fluid is hot saline to thermally ablate the organ. In another implementation the ablating fluid chemically ablates the organ through chemical oxidation. In another implementation the ablating fluid is electrified by, e.g., advancing an electrifying wire through the infusion lumen 30 and infusion port 32 of the ablation catheter 21 and energizing the wire using the source 18 of ablation energy shown in FIG. 1 to ablate the gall bladder. Yet again, the ablating fluid may be a gas such as a noble gas, and the gas can be electrified in accordance with above principles to ablate the organ. The ablating fluid alternatively may be Silver Nitrate or any other oxidizing fluid. Instead of fluid a diode or laser fiber can be advanced through the catheter and energized to ablate the gall bladder or appendix. The ablated organ can then be removed as described further below or it can be left in situ as a non-functional organ.

FIG. 4 shows an alternate ablation catheter 50 that in all respects may be identical to the ablation catheter 21 shown in FIGS. 2 and 3 with the following exceptions. The ablation catheter 50 shown in FIG. 4 can include an ablation balloon 52 on the distal end of the catheter 50 as shown. The ablation balloon 52 in a deflated configuration (not shown) is advanced into the gall bladder and then inflated with fluid such as saline, without the fluid emerging from the ablation balloon 52, such that the balloon 52 substantially fills the gall bladder and is urged against the walls of the gall bladder, slightly distending it. Then, bipolar electricity is applied to the fluid within the balloon in accordance with principles above to ablate the gall bladder. The catheter 50 may be used in similar manner to ablate the appendix.

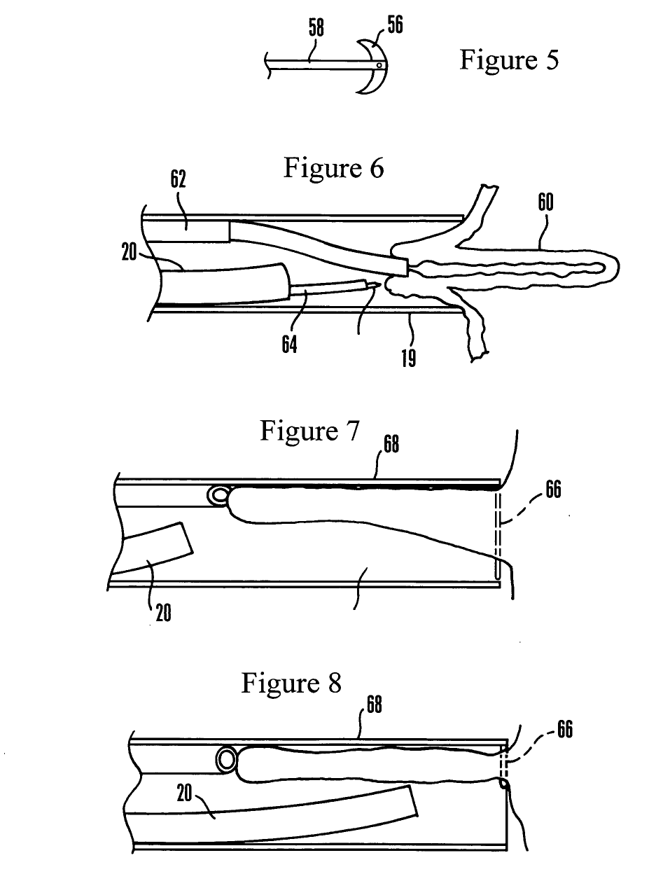

After ablation, in some implementations the organ can be inverted and ligated. In one non-limiting embodiment and referring back to FIG. 3 as an example, this may be accomplished by drawing a vacuum through the vacuum port 42 of the ablation catheter 21 to urge the gall bladder against the catheter, and then withdrawing the catheter 21, inverting the gall bladder. In another implementation, the ablation catheters of FIG. 3 or 4 can be removed and the now-desiccated organ can be inverted if desired using graspers 56 shown in FIG. 5, which can attached to a grasper shaft 58 that, e.g., is advanced through the working lumen of the endoscope 20. The graspers 56 can be rearwardly-arcing sharp prongs as shown to grip the desiccated tissue and invert it as the shaft 58 is withdrawn from the patient.

Still further alternate inversion structure may be used as shown in FIGS. 6-8, which for illustration show an appendix being inverted, it being understood that present principles also apply to inverting the gall bladder. With the overtube 19 advanced through a natural orifice to the organ, an inversion catheter 60 can be extended into the organ as shown in FIG. 6. A sealing sleeve 62 that slidably supports the inversion catheter 60 and that is slidably disposed in the overtube 19 is also advanced to the opening of the organ and may extend slightly into the organ as shown for providing a vacuum seal. The above operations can be visualized using the endoscope 20 disclosed above and/or by using fluoroscopy or ultrasonic imaging as set forth previously. A hollow cautery dissector catheter 64 can be advanced through the endoscope 20 for purposes to be shortly disclosed.

Once the inverter catheter 60 is positioned in the organ, structure on the catheter 60 urges the organ against the inverter catheter 60 so that upon proximal retraction of the inverter catheter 60 the organ inverts upon itself. Such structure can include vacuum holes that communicate with a vacuum lumen of the catheter 60 so that when a vacuum is drawn in the lumen, the organ is drawn against the catheter 60, with the sealing sleeve 62 functioning to prevent loss of vacuum within the organ.

FIG. 12 illustrates inversion of the organ such as an appendix caused by retracting the inverter catheter 60. As shown, a ligating cord 66 circumscribes that open distal end of the overtube 19 to form a loop. The cord 66 extends out of a flexible ligating catheter 68 in the overtube 19, it being understood that the proximal end of the cord 66 outside the patient can be pulled to tighten the loop.

It may now be appreciated that as the inversion catheter 60 is withdrawn in the overtube 19 the organ is inverted upon itself and is drawn into the overtube 19, with the loop of the ligating cord 66 surrounding the inverted organ. After the organ is inverted into the overtube 19, the loop of the ligating cord 66 is tightly cinched around the organ by appropriate pulling on the cord 66. If desired, the overtube 19 may be slightly retracted from the appendix at this point.

As understood herein, to facilitate inverting the appendix or gall bladder as additional tissue is being moved proximally, it may be necessary to use the cautery dissector catheter 64 to cut through the tissue being drawn into the overtube 19 to permit complete inversion by allowing the appendix to fully invert. The inverted, ligated organ may be left in the body to slough off and pass through the bowels, or it subsequently may be transected. Such transection methods and apparatus are disclosed in the assignee's co-pending U.S. patent application Ser. No. 11/601,199, filed Nov. 17, 2006, incorporated herein by reference. As mentioned above, an ablated inverted organ may be left in situ as a non-functional organ.

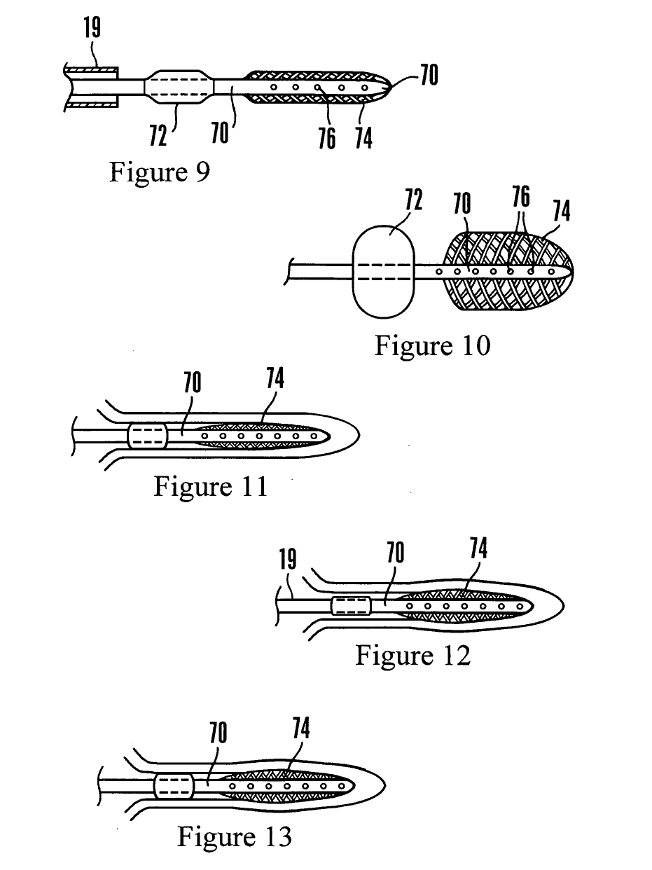

FIGS. 9-13 show an alternate ablation catheter generally designated 70 that in all essential respects is identical to the ablation catheters shown and described above with the following exceptions. Like the ablation catheter 21 shown in FIG. 3, the ablation catheter 70 shown in FIGS. 9-13 includes an inflatable anchoring balloon that can be positioned in the bile duct in a deflated configuration (FIG. 9) and then inflated (FIG. 10) to engage and substantially completely block the bile duct.

The ablation catheter 70 shown in FIGS. 9-13 includes on its distal end an expandable metal mesh or array 74 that has a collapsed configuration, shown in FIG. 9, wherein the mesh or array 74 can be advanced into an organ such as the gall bladder, and an expanded configuration, shown in FIG. 10, wherein the mesh or array 74 substantially completely fills the organ and urges against the walls of the organ, possibly slightly distending the organ. The mesh or array 74 can be an expandable metal such a nitinol that may be activated to expand in accordance with nitinol principles known in the art. To heat or energize the mesh or array 74 an electrical lead can be disposed in the catheter 70 and connected to a source of energy such as the source 18 shown in FIG. 1. Or, an inflatable balloon can reside within the mesh or array 74 and can be inflated and deflated in accordance with principles above to expand and collapse the mesh or array 74. Vacuum holes 76 may be formed in the catheter 70 and connected to a source of vacuum in accordance with prior principles.

With this structure, the mesh or array 74 in the collapsed configuration (and with the anchor balloon 72 deflated) can be advanced through a natural orifice through the overtube 19 into, e.g., the gall bladder or appendix (FIG. 11) and then expanded (FIG. 12). The overtube 19 may be withdrawn at the this point. Then, the anchor balloon 72 is inflated (FIG. 13) and the organ evacuated against the mesh or array 74 by drawing a vacuum through the vacuum holes 76 to further engage the organ with the mesh or array 74. The mesh or array 74 is then energized for, e.g., twenty to thirty seconds using, e.g., monopolar or bipolar current from the source 18 of energy shown in FIG. 1 to ablate the organ, or by directly heating the array to in essence establish a thermal array. It is to be understood that bipolar leads can be disposed through the catheter 70 to connect the source with the mesh or array 74. The ablated organ may then be inverted and ligated if desired in accordance with principles above.

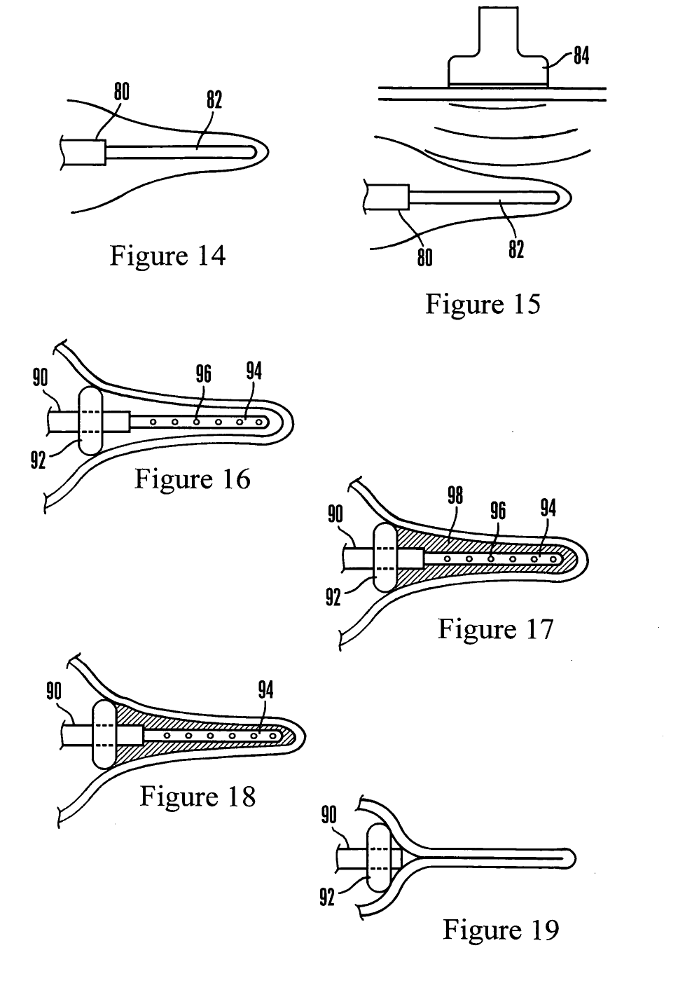

FIGS. 14 and 15 show an alternate ablation catheter 80 that can be advanced through a natural orifice through the overtube 19 or endoscope 20 or other structure into the intestines to ablate an organ such as the appendix or gall bladder. As shown, the catheter 80 includes an ablation member that is established by a high intensity focused ultrasound (HIFU) antenna 82. With the antenna 82 positioned in the organ as shown, a HIFU transmitter 84 can be positioned outside the patient against the abdominal wall and activated to transmit HIFU energy to the antenna 82, ablating the organ in a precise and controlled process. The organ subsequently may be ligated and/or transected or simply left in place to slough off.

FIGS. 16-19 show an alternate catheter 90 that in effect, like the ablation catheters shown and described above, is a natural orifice neutralization catheter, except that the catheter 90 shown in FIGS. 16-19 neutralizes an organ such as the appendix or gall bladder not by ablating it but by filling it with sealant.

With more specificity, the catheter 90 may include an anchoring balloon 92 that is inflatable inside the bile duct or just outside the appendix, and a sealant infusion tube 94 extending distally for advancement into the organ sought to be neutralized. Infusion holes 96 are formed in the infusion tube 94. The infusion holes 96 communicate through an infusion lumen of the catheter 90 with a source of adhesive outside the patient. The adhesive can be cyanocrylate glue. The infusion holes 96 may also communicate with the source 14 of vacuum shown in FIG. 1, or separate vacuum holes (that can communicate with the source 14 through a dedicated vacuum lumen in the catheter 90) may be provided.

With the above description in mind, with the anchor balloon 92 deflated, the infusion tube 94 is advanced into an organ such as the appendix (FIG. 16) and the anchor balloon 92 inflated. Adhesive 98 (FIG. 17) is then infused into the organ through the holes 96, filling the appendix with adhesive that, owing to the balloon 92, is held within the organ, effectively neutralizing it. Vacuum may then be drawn through the catheter 90 (FIG. 18) if desired to collapse the appendix as much as possible (FIG. 19). The catheter 90 is then removed. The organ may be left in situ if desired as an inert organ.

The distal cystic duct can be closed using glue and vacuum as well.

In another embodiment recognizing that an adhesive membrane is between the gall bladder and the inferior portion of the liver, a small hydrodissector or inflatable balloon similar to those described above with a means (laser, chemical, electrical, or mechanical) for inducing hemostasis can be advanced into the gall bladder through a small incision in the membrane. Fluid or mechanical means can be used to dissect the gall bladder from the liver bed while applying hemostasis. The gall bladder is inverted on itself as described above and morcelated from within the bile duct, thus eliminating the need to enter the abdominal space.

While the particular SYSTEMS AND METHODS FOR LESS INVASIVE NEUTRALIZATION BY ABLATION OF TISSUE INCLUDING THE APPENDIX AND GALL BLADDER is herein shown and described in detail, it is to be understood that the subject matter which is encompassed by the present invention is limited only by the claims.

What is claimed is:

1. A method comprising:
advancing a catheter assembly into the intestines of a patient through a natural orifice of the patient;
advancing a neutralization element of the catheter assembly into an organ of the patient; and
actuating the neutralization element to neutralize the organ from the interior thereof.

2. The method of claim 1, wherein the neutralization element is an ablation catheter, and an ablating fluid is infused into the organ through the ablation catheter.

3. The method of claim 2, wherein the ablating fluid is hot to thermally ablate the organ.

4. The method of claim 2, wherein the ablating fluid chemically ablates the organ.

5. The method of claim 2, comprising electrifying the ablating fluid to ablate the organ.

6. The method of claim 2, wherein the ablating fluid is a gas, and the method comprises electrifying the gas to ablate the organ.

7. The method of claim 2, wherein the ablation catheter includes a balloon, the organ is the gall bladder, and the method comprises advancing at least a portion of the catheter into the gall bladder with the balloon in the bile duct and inflating the balloon to seal the ablating fluid in the gall bladder.

8. The method of claim 2, wherein the ablating fluid is Silver Nitrate.

9. The method of claim 1, wherein the neutralization element is an ablation catheter having an expandable ablation member configured for ablating the organ.

10. The method of claim 9, wherein the ablation member is a balloon inflatable with a fluid to substantially fill the organ, the fluid being electrified to ablate the organ.

11. The method of claim 9, wherein the ablation member is an expandable metal mesh or array and the method includes electrifying the mesh or array to ablate the organ.

12. The method of claim 1, wherein the neutralization element is an antenna and the method comprises transmitting, from outside the patient, high intensity focused ultrasound (HIFU) energy to the antenna to ablate the organ.

13. The method of claim 1, wherein the neutralization element is an adhesive infusion tube and the method comprises infusing adhesive into the organ through the tube and then applying vacuum to the organ to collapse it.

14. The method of claim 1, wherein prior to ablating the organ the method comprises destroying a mucus membrane of the organ.

15. A method comprising:
advancing a catheter assembly into the intestines of a patient through a natural orifice of the patient;
advancing a sealant element of the catheter into an organ of the patient; and
infusing sealant through the sealant element into the organ to seal the organ.

16. The method of claim 15, wherein the sealant is cyanocrylate glue and the organ is the appendix.

17. The method of claim 15, wherein the sealant element is a sealant catheter having a balloon, and the method comprises advancing at least a portion of the sealant catheter into the organ and inflating the balloon to seal the sealant in the organ.

18. The method of claim 15, comprising evacuating the organ after infusing sealant.

19. The method of claim 18, comprising withdrawing the catheter and leaving the organ in situ as an inert organ without removing the organ.

20. A system for ablating the gall bladder or appendix, comprising:
a catheter assembly advanceable into the intestines of a patient through a natural orifice of the patient;
an ablation catheter advanceable out of the catheter assembly into the gall bladder or appendix; and
means engaged with the ablation catheter for ablating the gall bladder or appendix.

21. The system of claim 20, wherein the means for ablating is an ablating fluid, the system comprising a source of ablating fluid engaged with the catheter assembly for infusing the ablating fluid through the ablation catheter into the gall bladder or appendix.

22. The system of claim 21, wherein the ablating fluid is hot to thermally ablate the gall bladder or appendix.

23. The system of claim 21, wherein the ablating fluid chemically ablates the gall bladder or appendix.

24. The system of claim 21, comprising a source of electricity engaged with the catheter assembly and energizable to electrify the ablating fluid to ablate the gall bladder or appendix.

25. The system of claim 21, wherein the ablating fluid is a gas, and the system includes a source of electricity engaged with the catheter assembly and energizable to electrify the gas.

26. The system of claim 21, wherein the ablation catheter includes a balloon inflatable to block the bile duct to seal the ablating fluid in the gall bladder.

27. The system of claim 21, wherein the ablating fluid is Silver Nitrate.

28. The system of claim 20, wherein the means for ablating is an expandable ablation member on the ablation catheter and configured for ablating the gall bladder or appendix.

29. The system of claim 28, wherein the ablation member is a balloon inflatable with a fluid to substantially fill the gall bladder or appendix, the fluid being electrified to ablate the gall bladder or appendix.

30. The system of claim 28, wherein the ablation member is an expandable metal mesh electrifiable to ablate the gall bladder or appendix.

31. A system for neutralizing the gall bladder or appendix, comprising:
a catheter assembly advanceable into the intestines of a patient through a natural orifice of the patient;
a sealant catheter advanceable out of the catheter assembly into the gall bladder or appendix; and
a source of sealant engaged with the catheter for infusing sealant through the catheter to seal the gall bladder or appendix.

32. The system of claim 31, wherein the sealant is cyanocrylate glue.
